(19) United States
(12) Patent Application Publication
(10) Pub. No.: US 2024/0358397 A1
(43) Pub. Date: Oct. 31, 2024

(54) Systems, Methods, and Apparatuses for Removing Materials and Plaque in Arteries and Blood Vessels

(71) Applicant: Opus Medtech LLC, Lake Forest, CA (US)

(72) Inventor: LE Brandon, Lake Forest, CA (US)

(21) Appl. No.: 18/648,192

(22) Filed: Apr. 26, 2024

Related U.S. Application Data
(60) Provisional application No. 63/461,952, filed on Apr. 26, 2023.
(60) Provisional application No. 63/611,711, filed on Dec. 18, 2023.

(51) Int. Cl. A61B 17/3207; A61B 17/12; A61B 17/22; A61B 90/00; A61M 25/10

(52) U.S. Cl. CPC A61B 17/320725; A61B 17/1204; A61B 17/12109; A61B 17/12136; A61B 2017/22038; A61B 2090/3966; A61M 2025/1013; A61M 2025/1052; A61M 2025/109

(57) ABSTRACT

A medical device includes a hub having an output port, a distal balloon inflation port, a proximal balloon inflation port, a distal balloon, a flexible inner inflation shaft with a proximal end coupled to the distal balloon inflation port and a distal end coupled to the distal balloon to carry a gas, liquid or solution that enters the distal balloon inflation port and flows to and inflates the distal balloon. The device further comprises a proximal balloon and a flexible outer inflation shaft coaxially aligned with the inner inflation shaft with a proximal end coupled to the proximal balloon inflation port and a distal end coupled to the proximal balloon to carry a gas, liquid or solution that enters the proximal balloon inflation port and flows to and inflates the proximal balloon.

### CLAIM OF PRIORITY

This U.S. Utility non-provisional patent application is related, and claims priority, to U.S. provisional patent application No. 63/611,711, filed Dec. 18, 2023, entitled “SYSTEMS AND METHODS FOR REMOVING UNDESIRABLE MATERIALS AND PLAQUE IN AUTOGENOUS ARTERIOVENOUS FISTULA (aAVF) AND CURVED ARTERIES AND VESSELS IN LOWER LEGS (DVT)”, having Attorney Docket Number 38512.602P, and is further related, and claims priority, to U.S. provisional patent application No. 63/461,952, filed Apr. 26, 2023, entitled “SYSTEMS AND METHODS FOR REMOVING UNDESIRABLE MATERIALS AND PLAQUE IN AUTOGENOUS ARTERIOVENOUS FISTULA (aAVF) AND CURVED ARTERIES AND VESSELS IN LOWER LEGS (DVT)”, having Attorney Docket Number P20230003, the entire contents of each being incorporated herein by reference as though set forth in full.

### TECHNICAL FIELD

Embodiments of the invention relate generally to the field of medical devices, and in particular to a dual-balloon catheter that can be inserted into an artery or vein according to various medical procedures.

### BACKGROUND

Blood clots that form in arteries (arterial clots) or veins (venous clots) can be serious. Blood clots are gel-like collections of blood that form in a body's veins or arteries when blood changes from liquid to partially solid. Clotting is a normal function that stops a body from bleeding too much when injured. However, blood clots that form in some places and do not dissolve on their own can be dangerous to a person's health. Thus, blood clots are a serious medical condition. Blood clots that form in the veins in a person's legs, arms, and groin can break loose and move to other parts of the body, including the lungs. A blood clot that forms in deep veins such as the veins of the legs, arms, or groin of a person is referred to as Deep Vein Thrombosis (DVT). A stationary blood clot, or one that stays in place, may not harm a person. However, a blood clot that dislodges and begins moving through one's bloodstream can be harmful. One of the most pressing blood clot concerns is when a DVT clot makes its way to one's lungs and remains lodged there. If this happens, one's life can be in danger.

Hemodialysis (H/D) is a common form of renal replacement therapy (RRT) for End Stage Renal Disease (ESRD) patients. To begin H/D, a patient must first establish a high blood flow and long-lasting vascular access. The autogenous arteriovenous fistula (aAVF, or simply, AVF) is the most common and efficient vascular access for hemodialysis. The AVF is created by mobilizing and transecting a superficial vein close to an artery and transposing (“swinging”) the vein towards the artery. An arteriovenous anastomosis is then created by suturing the vein, end to side, to the artery. Arterial flow will then enter the vein providing adequate blood flow for H/D.

Arterial flow into the vein will cause a physiologic arterialization of the vein. This physiologic response is present throughout the lifespan of the AVF. Arterialization of the vein causes intimal hyperplasia (INH), causing a thickening of the intima. This is most prominent at the peri-anastomotic vein and artery (the “swing” site). The INH will decrease the lumen diameter, causing stenosis, i.e., a narrowing, of the inflow artery and outflow vein. This will result in decreased blood flow to the vein and inadequate blood flow for adequate hemodialysis. Further increase in stenosis will finally result in total occlusion and thrombosis (where a blood clot blocks a vein or artery) of the AVF. This is a major cause of abandonment of an AVF.

Presently, the standard treatment of stenoses in the access circuit is through balloon angioplasty of the lesions (POBA). However, due to the disparity of lumen diameters of the peri-anastomotic artery and vein, two balloon catheters are used to dilate the lesions present in both vessels. The present balloon geometry also caused “straightening” of the anastomosis causing further injury to the intima and in the most severe cases, rupturing the anastomosis.

What is needed is a dual-balloon catheter device that can be used in these AVF and DVT scenarios, as well as peripheral vasculature or vasculature where two balloons may be used.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1A illustrates a medical device according to an embodiment of the invention;

FIG. 1B illustrates a transparent view of a medical device according to an embodiment of the invention;

FIGS. 2A, 2B, 2C, 2D and 2E illustrate aspects of balloons coupling to respective inflation shafts according to an embodiment of the invention;

FIG. 3A illustrates a medical device according to an embodiment of the invention in which the balloons are not inflated;

FIG. 3B illustrates a medical device according to an embodiment of the invention in which the balloons are inflated;

FIGS. 4A, 4B, 4C and 4D illustrate embodiments of the invention in which one or more scoring wires are installed on an outer surface of one or both balloons of a medical device; and

FIGS. 5A and 5B illustrate embodiments of the invention that can be used for dialysis treatments.

### DETAILED DESCRIPTION

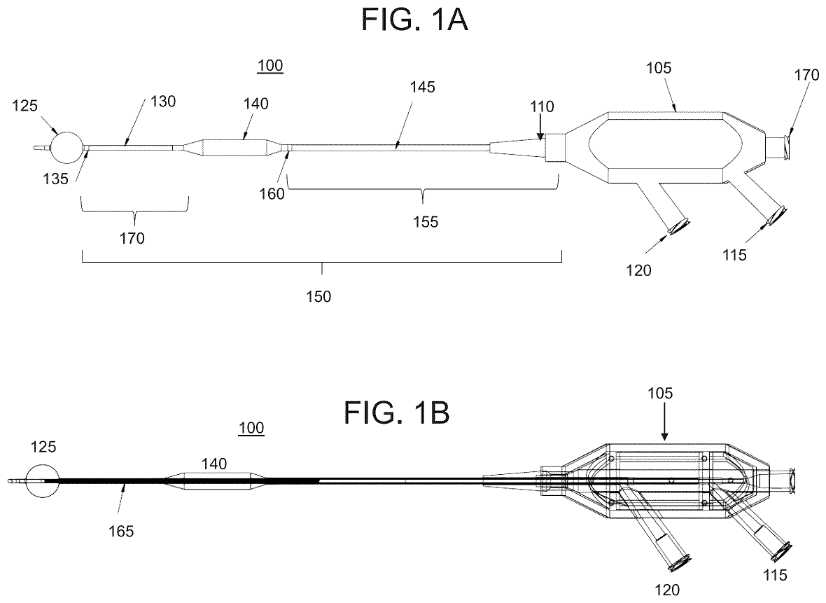

FIGS. 1A and 1B illustrate a medical device 100, referred to herein as a dual-balloon catheter, that may be used in treating, for example, Deep Vein Thrombosis (DVT), autogenous arteriovenous fistula (AVF), as well as health issues involving peripheral vasculature of the human body. With reference to FIGS. 1A and 1B, the medical device 100 comprises a hub 105 having an output port 110, a distal balloon inflation port 115, and a proximal balloon inflation port 120. While the illustrated embodiment shows both port 115 and 120 on the same side of the hub 105, it is appreciated that the ports can be positioned on opposite or adjacent sides of the hub, so long as each has a respective separate connection to inflation shafts that carry a fluid, gas, liquid or solution to a separate inflatable balloon, as described in detail below. According to one embodiment, the hub 105 may be a multi-Y-arm connector hub, e.g., a two-arm connector hub, or a two-arm Leur connector hub, that connects via one or more input ports to one or more sources of a gas (e.g., air) or a liquid or a solution (e.g., saline, or a combination of a gas and a liquid), or a source of blood that has been removed from the body and treated, e.g., cleaned for harmful substances, before being returned to the body, for example, as clean blood. According to one embodiment a saline solution comprising 50% (or thereabout) saline is combined with 50% (or thereabout) high contrast liquid such as iodine so a physician can see in an ultrasound the location and state of the balloon.

According to an embodiment, medical device 100 is a dual-balloon catheter medical device. The medical device includes a balloon 125 referred to herein as the distal balloon 125 given it is further away from the hub 105 than balloon 140. (All references herein to “distal”, whether to the “distal balloon”, or to some other distal element or component, indicate the distance from the “distal” balloon or element to hub 105 as being longer than the distance from the “distal” balloon or element to hub 105, and therefore farther away from, or distal with respect to, hub 105). The distal balloon 125 is illustrated in its inflated state, whereby it can occlude blood flow in a vein or artery in a body of a patient. A flexible inner inflation shaft or tube 130 having a first length 150 includes a proximal end coupled through output port 110 of hub 105 to distal balloon inflation port 115 and a distal end 135 coupled to the distal balloon 125. In one embodiment, the proximal end is coupled through the output port 110 of the hub 105 to the distal balloon inflation port 115 via a first, isolated, internal chamber. The inner inflation shaft 130 can carry a gas, liquid or solution that enters the distal balloon inflation port 115 of hub 105 and flows from the hub's distal balloon inflation port 115 to the distal balloon 125 to inflate the distal balloon.

The medical device further includes a balloon 140, referred to herein as the proximal balloon 140 because it is closer to the hub 105 than balloon 125. (All references herein to “proximal”, whether to the “proximal balloon”, or to some other proximal element or component, indicate the distance from the “proximal” balloon or element to hub 105 as being shorter than the distance from the “distal” balloon or element to hub 105, and therefore closer to, or proximal or proximate with respect to, hub 105). The proximal balloon is illustrated in its inflated state, where it can engage or contact material with a vein or artery, such as a blood clot or plaque, or implanted material such as a stent, but otherwise allow blood to flow through the vein or artery. A flexible outer inflation shaft or tube 145 surrounds and is coaxially aligned with the inner inflation shaft 130. The length 155 of the flexible outer shaft is shorter than the length 150 of the inner inflation shaft. A proximal end of the outer inflation shaft is also coupled through output port 110 of hub 105 to proximal balloon inflation port 120, and a distal end 160 of the outer inflation shaft is coupled to the proximal balloon 140. In one embodiment, the proximal end is coupled through the output port 110 of the hub 105 to the proximal balloon inflation port 120 via a second internal chamber separate and isolated from the first internal chamber of hub 105. The outer inflation shaft can carry a gas, liquid or solution that enters the proximal balloon inflation port 120 of hub 105 and flows through the hub's output port 110 to the proximal balloon 140 to inflate the proximal balloon, independently of and separately from the inflation of distal balloon 125 which happens, as discussed above when the inner inflation shaft 130 carries a gas, liquid or solution that enters the distal balloon inflation port 115 of hub 105 and flows through the hub's output port 110 to the distal balloon 125 to inflate the distal balloon.

While the embodiments illustrated herein depict the distal balloon 125 in an inflated state having an outer diameter that is greater than outer diameter of the proximal balloon 140 in an inflated state, it is appreciated that depending on the treatment being performed and a specific point in time at which the treatment is being performed, that the outer diameter of the distal balloon may be less than, greater than, or equal to the outer diameter of the proximal balloon. Moreover, while the shape of the distal balloon when inflated to create occlusion in a vein or artery is depicted as generally spherical and the shape of the proximal balloon when inflated is depicted as generally columnar or cylindrical, it is appreciated that either balloon may be either shape depending on the treatment being performed. Furthermore, while the length (e.g., diameter) of the distal balloon appears to be shorter than the length of the proximal balloon along the longitudinal axis of the medical device, it is appreciated that in some embodiments, the length of the distal balloon along the longitudinal balloon may be greater than or equal to the length of the proximal balloon, again, depending on the context of treatment being performed. Additionally, the proximal and distal ends of each balloon with respect to the longitudinal axis of the medical device may be accompanied by a respective pair of markers that are visible to practitioners so that they can position each balloon at appropriate locations within a vein or artery for treatment. For example, the markers may comprise a radioplaque material (whether metallic markers, or polymeric markers with radioplaque powder embedded therein) that makes the markers visible in an X-ray or via fluoroscopy, and thereby indicate where a balloon is located. This is particularly important when the balloons are not inflated and their outside diameters may be the same or similar to the outside diameter of the inflation shaft to which they are coupled.

Inflation of one or more of the distal balloon 125 and the proximal balloon 140 while the medical device is inserted in a vein or artery of a body can cause damage, such as cracks or tears in the intima layer of the vein or artery. Thus, in one embodiment, one or both balloons may be coated with appropriate drugs that assist in healing damage or tears to the vein or artery.

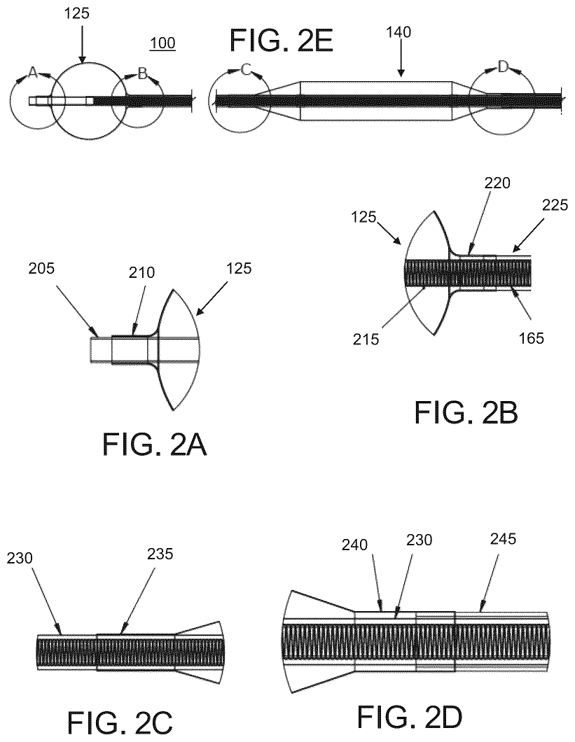

FIGS. 2A-2E illustrate details of how the distal balloon 125 and proximal balloon 140 are coupled to the respective inner inflation shaft and outer inflation shaft, according to embodiments of the invention. FIG. 2E illustrates pertinent portions of the medical device 100, including the distal balloon 125 and the proximal balloon 140 and the connection points between the proximal and distal ends of each balloon to the respective inner and outer inflation shafts. These connection points are circled and labeled A, B, C and D in FIG. 2E, and correspond to FIGS. 2A, 2B, 2C and 2D, that provide a better view of the connection points.

For example, FIG. 2A illustrates a distal leg 210 of distal balloon 125 coupled, e.g., fused, bonded, clamped or adhered (e.g., using heat shrinking or an adhesive such as glue), to a distal, terminal, portion of the coil 165 or guidewire, or an innermost shaft or tube that houses the coil or guidewire, as depicted at 205. This coil, or guidewire, or innermost shaft, extends from the guidewire port 170 at the proximal end of the medical device to the distal tip or end of the medical device. The distal tip or end is open. Thus, depending on blood pressure, blood may flow from the distal tip back to the hub 105, or the guidewire port 170 of hub 105, along the coil, guidewire or innermost shaft. In some embodiments, a hemostatic valve at hub 105 can be installed to prevent blood loss due to blood flowing from the distal tip to hub 105.

While the embodiment depicts the distal tip of the coil, guidewire, or innermost shaft that houses either the coil or guidewire as perpendicular, i.e., a 90 degree angle with respect, to the longitudinal axis of the medical device, in other embodiments the distal tip may be slanted at a greater than 90 degree angle with respect to the longitudinal axis of the medical device to facilitate insertion of the distal tip and subsequent elements of the medical device, such as the distal balloon, inner and outer inflation shafts, or the proximal balloon, into a vein or artery. In various embodiments, the outside diameter of the coil may be smaller than the outside diameter of either balloon, for example, the outside diameter of the coil can be in the range of ⅓- 1/15 of an inch in outside diameter. Thus, the inner diameter of the innermost shaft is also much smaller than either balloon, for example, the inner diameter of the innermost shaft can be in the range of ⅓- 1/15 of an inch. Similarly, FIGS. 2A and 2B illustrates a proximal leg 220 of distal balloon 125 coupled to a distal, terminal portion 225 of inner inflation shaft 130, 230. In this manner, a fluid, gas, liquid or solution can be carried through inner inflation shaft 130, 230 to distal balloon 125 to inflate distal balloon 125. Also depicted is the coil 165 or innermost tube housing the coil entering the distal balloon 125. Alternatively, or additionally, a guidewire may enter distal balloon 125, or an innermost tube with guidewire inside it may enter distal balloon 125. Note coil 165 and/or guidewire, or innermost tube housing either of the coil or guidewire, may extend through distal balloon 125 and past the distal leg 210 of distal balloon 125 as depicted in FIG. 2A to provide structural support for distal balloon and to assist in inserting, positioning, and retracting the combination of the distal balloon 125, inner inflation shaft 130, proximal balloon 140 and outer inflation shaft with respect to an artery or vein of the patient.

FIG. 2C illustrates a distal leg 235 of proximal balloon 140 coupled to a portion of inner inflation shaft 230. Note outer inflation shaft 140, 240 does not extend to the distal leg of proximal balloon 140. FIG. 2D illustrates a proximal leg 240 of proximal balloon 140 coupled to a distal, terminal, portion of outer inflation shaft 245. Note inner inflation shaft 130, 230 extends past the proximal leg of proximal balloon 140 and continues through an inner chamber of proximal balloon 140 and beyond to distal balloon 125. In this manner, a gas, solution or liquid can be carried through outer inflation shaft 140, 240 to proximal balloon 140 to inflate proximal balloon 140 and a different source of a gas, solution or liquid (whether the same or a different gas, solution or liquid) can be carried through inner inflation shaft 130, 230 to distal balloon 125 to inflate distal balloon 125.

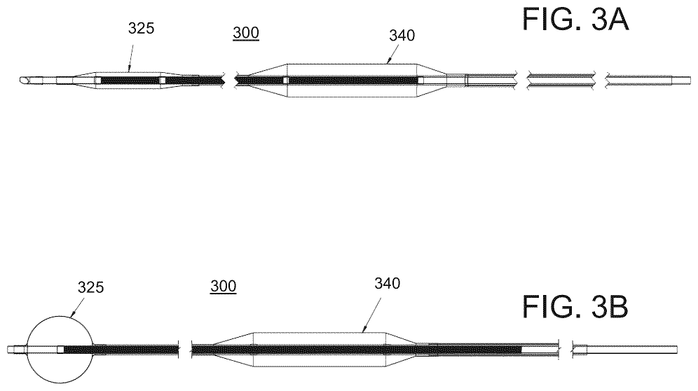

FIG. 3A illustrates an embodiment 300 in which distal balloon 125, 325 is in its uninflated state, whereby it does not occlude blood flow in a vein or artery in a body of a patient. Rather, it can engage or contact material with a vein or artery, such as a blood clot or plaque, or implanted material such as a stent, but otherwise allow blood to flow through the vein or artery. Likewise, the proximal balloon 140, 340 is illustrated in its inflated state, where it can engage or contact material with a vein or artery, such as a blood clot or plaque, or implanted material such as a stent, or open or expand a vein or artery, but otherwise allow blood to flow through the vein or artery. FIG. 3B illustrates the embodiment 300 in which distal balloon 125, 325 is in its inflated state, whereby it occludes blood flow in a vein or artery in a body of a patient. The proximal balloon 140, 340 is illustrated in its inflated state, where it can engage or contact material with a vein or artery, such as a blood clot or plaque, or implanted material such as a stent, or open or expand an artery or vein, but otherwise allow blood to flow through the vein or artery.

Returning to FIGS. 1A and 1B, according to an embodiment of the medical device 100, the distal balloon 125 is positioned a third distance 170 from the proximal balloon 140 since the second length 155 of the flexible outer inflation shaft 145 is shorter than the first length 150 of the flexible inner inflation shaft 130. In an embodiment, the third distance is a fixed distance, for example, determined at the time of manufacture of the medical device. In another embodiment, the third distance is configurable by a user, e.g., a physician about to perform a medical treatment on a patient. Thus, a segment of the flexible inner inflation shaft 130 extends a third distance 170 by itself-not surrounded by the outer inflation shaft, between the proximal and distal balloons. This segment of the flexible inner inflation shaft, or at least a portion thereof, allows for the flexible inner inflation shaft to bend or shift or transition with respect to the position of the proximal balloon, for example, in a curved, U-, or S-shaped configuration as the distal balloon, the flexible inner inflation shaft to which the distal balloon is coupled, and the proximal balloon traverse and/or are positioned within a curved vein or artery of the body, or a junction between veins, arteries, or a vein and artery, in the body.

In one embodiment, the segment of the flexible inner shaft 130 that extends between the distal side of the proximal balloon and the proximal side of the distal balloon may be accompanied by one or a pair of markers (not shown in the figures) that are visible to practitioners so that they can position the segment at appropriate locations within a vein or artery for treatment, for example, where the segment traverses or is positioned within a curved portion of a vein or artery, or at a junction between veins or arteries. For example, the marker(s) may comprise a radioplaque material that makes the marker(s) visible in an X-ray or via fluoroscopy, and thereby indicate where a bendable segment, or bendable portion thereof, is located.

According to an embodiment, the medical device 100 includes a coil 165 coaxially aligned with the flexible inner inflation shaft 130. According to one embodiment, the coil provides structural support to assist in inserting, positioning, and retracting the combination of the proximal and distal balloons with respect to a vein and/or artery of the body. According to an embodiment, the coil comprises a radioplaque material. A radioplaque material is opaque to one or another form of radiation or ionizing radiation such as X-rays or flouroscope. Radiopaque objects block radiation rather than allow it to pass through. Metal, for instance, is radiopaque, so metal objects that are in a patient are visible on X-rays. In one embodiment, the radioplaque material comprises platinum, iridium, or gold. In another embodiment, the coil comprises a polymer material combined with powders that are radioplaque.

According to an embodiment, the medical device 100 can receive and use a guidewire to assist in inserting, positioning, and retracting the combination of the proximal and distal balloons with respect to a vein and/or artery of the body. In such an embodiment, the hub 105 further comprises a guidewire port 170, through which the guidewire can be inserted to pass through the hub 105 and the hub's output port 110 and extend inside along the length of flexible inner inflation shaft 130. In one embodiment, both the coil 165 and guidewire may be used, for example, with the guidewire coaxially aligned and inside the coil. In another embodiment, the guidewire is by itself, inside and coaxially aligned with the flexible inner inflation shaft 130. According to one embodiment, the guidewire can provide structural support to assist in inserting, positioning, and retracting the combination of the proximal and distal balloons with respect to a vein and/or artery of the body. According to an embodiment, the guidewire comprises a radioplaque material.

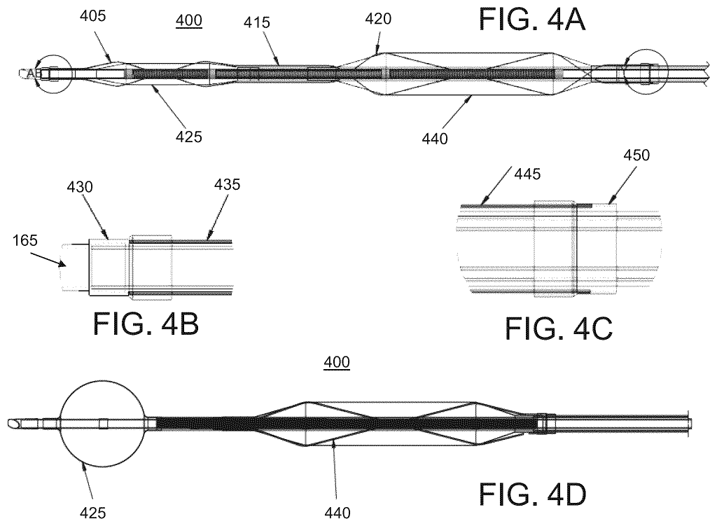

With reference to FIGS. 4A, 4B, 4C and 4D, according to an embodiment 400, the medical device 100 further comprises one or more scoring elements or wires (depicted at 405, 420) affixed to an outer surface of one or both of the distal balloon 125 and the proximal balloon 140 so that when a gas, solution or liquid inflates one or both of the distal balloon and the proximal balloon, the one or more scoring wires engages and cuts through or scrapes away undesirable material such as a clot or plaque or implanted material such as a stent in a vein or artery in which the distal balloon and proximal balloon is situated. In one embodiment, the one or more scoring elements or wires form a mesh or “fishnet” of such elements or wires over the outer surface of one or both of the distal balloon and proximal balloon. In another embodiment, the one or more scoring elements or wires spiral across the outer surfaces of one or both balloons, and/or the outer surface of the segment of inner inflation shaft located between the two balloons.

In one embodiment, the scoring elements or wires may be folded or pleated around the outer surface of one or both of the distal balloon and the proximal balloon, for example, in a clockwise direction about the longitudinal axis of the medical device, so that the edges of the scoring elements lay down flat along the surface and do not engage a vein or artery when the balloons are inserted in a vein or artery. Once the balloons are in a desired location, for example, positioned in an area of the vein or artery where unwanted material such as plaque or a clot is present, or where implanted material such as a stent is present, the medical device can be rotated in the opposite direction thereby unfolding the edges of the scoring elements, causing them to twist, stand up, and engage the unwanted or implanted material and cut or scrape such from its attachment or where it adheres to the intima, or inner lining, of the vein or artery.

Details of pertinent circled portions AB and Y in FIG. 4A are illustrated in FIGS. 4B and 4C. In FIG. 4B legs 435 of scoring element or wires 405, 420 are affixed at 430 to coil 165 or guidewire, or an innermost tube that houses the coil or guidewire, for example, via a heat shrink tubing coaxially aligned with coil 165 or a guidewire or innermost tube, coterminous with the distal legs of distal balloon 125. In FIG. 4C, legs 445 of scoring element or wires 405, 420 are affixed at 450 to outer inflation shaft 145 coterminous with proximal legs of proximal balloon 140. In this embodiment the scoring elements or wires 405, 420 traverse the entire length of the distal and proximal balloons including the outer surface 415 of the portion of the flexible inner inflation shaft between the distal balloon and the proximal balloon. In another embodiment, one or more scoring elements or wires 405 may only encompass or surround the outer surface of the distal balloon 125, 425 while another one or more scoring elements or wires 420 may only encompass or surround the outer surface of the proximal balloon 140, 440. In some embodiments, the scoring element or wires comprise an alloy of nickel titanium (nitinol) material. Nitinol is a nearly equi-atomic metal alloy of nickel and titanium with unique properties, including super elasticity (also called pseudo-elasticity) and shape memory. Super elasticity/pseudo-elasticity means nitinol shows great elasticity under stress and can snap back to its original shape when pressure is released. Thus, it is a useful material for the scoring elements or wires that surround the distal and proximal balloons so that as the balloons repeatedly move from a non- or un-inflated state to an inflated state, for example, as distal balloon 425 is depicted in FIG. 4D, the wires maintain or parallel the shape of the balloons. In other embodiments, the scoring element or wire may comprise any metallic material, a composite material, or a polymer material.

Recall FIG. 3B illustrates the embodiment 300 in which distal balloon 125, 325 is in its inflated or expanded state, whereby it occludes blood flow in a vein or artery in a body of a patient in which the distal balloon is lodged. It is contemplated that scoring may be performed according to any one of the above-described embodiments while the distal balloon is in the expanded state and occluding blood flow. Such scoring may result in unwanted or implanted material or debris, such as plaque, a stent, or portions thereof, in the blood pooled behind the location of the distal balloon, i.e., blood pooled between the inflated distal balloon and the proximal balloon or between the distal balloon and the point of insertion of the medical device into the vein or artery. Thus, one way to remove this unwanted or implanted material is to maintain the distal balloon in the expanded or inflated state so that it continues to occlude blood flow beyond, i.e., in front of, the distal balloon, and while the distal balloon is maintained in the expanded or inflated state, retract the distal balloon from the vein or artery all the way to the insertion point and thereby remove or flush the unwanted material or debris. This is a particularly useful process when clearing plaque or clots in the carotid artery to prevent debris from flowing to the brain, risking a stroke.

In an alternative embodiment, proximal balloon 140, 340 is in its inflated or expanded state, whereby it occludes blood flow in a vein or artery in a body of a patient in which the proximal balloon is lodged. It is contemplated that scoring may be performed according to any one of the above-described embodiments while the proximal balloon is in the expanded state and occluding blood flow. Such scoring may result in unwanted or implanted material or debris, such as plaque, a stent, or portions thereof, in the blood pooled in front of the location of the proximal balloon, i.e., blood pooled between the inflated proximal balloon and the distal balloon or between the proximal balloon and the point of insertion of the medical device into the vein or artery. Thus, one way to remove this unwanted or implanted material is to maintain the proximal balloon in the expanded or inflated state so that it continues to occlude blow flood beyond, i.e., behind, the proximal balloon, and while the proximal balloon is maintained in the expanded or inflated state, retract the proximal balloon from the vein or artery all the way to the insertion point and thereby remove or flush the unwanted material or debris.

According to the above described embodiments, depending on the patient health scenario, a portion of the medical device including the distal balloon, proximal balloon, and inner and outer inflation shafts, can be inserted into a vein or artery of the patient, wherein the inner inflation shaft 130 can carry a gas, solution or liquid that enters the distal balloon inflation port 115 of hub 105 and flows through the hub's output port 110 to the distal balloon 125 to inflate the distal balloon. Likewise, the outer inflation shaft 145 can carry the gas, solution or liquid that enters the proximal balloon inflation port 120 of hub 105 and flows through the hub's output port 110 to the proximal balloon 140 to inflate the proximal balloon. These two events or operations can occur at simultaneous, concurrent, overlapping, or non-overlapping time periods, according to the treatment needs of the patient.

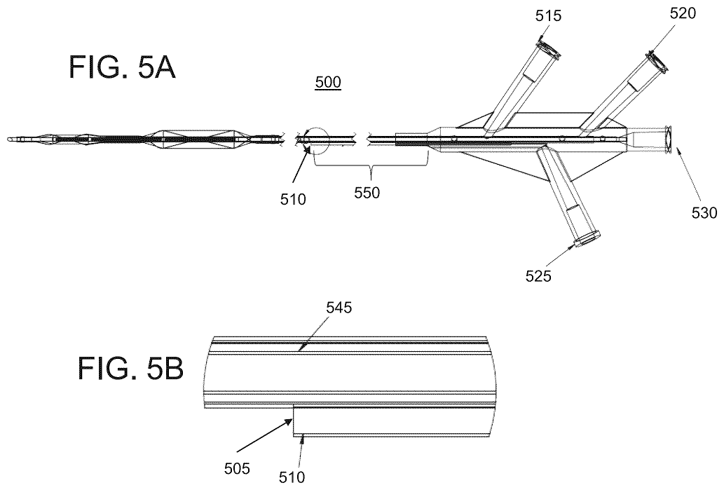

FIGS. 5A and 5B illustrate an embodiment 500 of the medical device 100 that includes a dialysis input port 525 and a dialysis output port 530 at hub 105, and a return blood flow shaft 510 affixed to an outer surface of the flexible outer inflation shaft 145, 545 having a third length 550 shorter than the second length 155 of the outer inflation shaft. The return blood flow shaft 510 has a return blow flow shaft output port 505 via which cleaned and/or oxygenated blood can be returned to the vein or from hub 105. Blood can be withdrawn from an artery through the innermost shaft coupled to dialysis output port 530 and then to a dialysis machine where the blood may be cleaned to remove harmful substances before being returned to the body as clean and/or oxygenated blood via dialysis input port 525, return blood flow shaft 510 and return blood flow shaft output port 505. This embodiment includes proximal balloon inflation port 115, 515 and distal balloon inflation port 120, 520 that each connect to a source of a gas, liquid or solution, such as a saline solution (or 50% saline and 50% high contrast liquid such as iodine so a physician can see in an ultrasound or ultrasound that location and state of the balloon) so that the inner inflation shaft 130 can carry a gas, liquid or solution that enters the distal balloon inflation port 115, 515 of hub 105 and flows through the hub's output port 110 to the distal balloon 125 to inflate the distal balloon, and the outer inflation shaft can carry a gas, liquid or solution that enters the proximal balloon inflation port 120, 520 of hub 105 and flows through the hub's output port 110 to the proximal balloon 140 to inflate the proximal balloon, according to the needs of a dialysis patient.

In one embodiment, dialysis output port 530 doubles as the guidewire input port 170. The distal balloon of the medical device is inserted into an artery of the body using a guidewire inserted through input port 170 and extending to the distal tip of the device, in front of the distal balloon, while the proximal balloon of the medical device remains in a vein of the body. Once the balloons of the device are in their respective locations to perform dialysis, the guidewire can be removed and a dialysis machine connected to dialysis port 530, where blood from the body can be withdrawn from an artery for cleaning by the dialysis machine and then the cleaned and/or oxygenated blood is routed back to vein in the body via dialysis input port 525, return blood flow shaft 510, and out return blood flow shaft output port 505 where it can continue to flow into the vein toward the heart. Advantageously, this embodiment allows for insertion of just one device for insertion and clearing of any obstructions in the artery and/or vein, followed with circulating blood from the artery through the dialysis machine for cleaning or treatment and back into a vein of the body.

While embodiments of the invention have been shown and described herein, those skilled in the art will appreciate that such embodiments are provided by way of example only. Variations and substitutions will occur to those skilled in the art without departing from the scope of the embodiments and such alternatives to the embodiments described herein may be employed in practicing the embodiments. It is intended that the following claims define the scope of the embodiments of the invention and that methods and structures within the scope of these claims and their equivalents are likewise covered.

What is claimed is:

1. A medical device, comprising:
a hub having an output port, a distal balloon inflation port, and a proximal balloon inflation port;
a distal balloon;
a flexible inner inflation shaft having a first length, a proximal end coupled to the distal balloon inflation port, and a distal end coupled to the distal balloon, the inner inflation shaft to carry a gas, liquid or solution that enters the distal balloon inflation port and flows through the output port to the distal balloon to inflate the distal balloon;
a proximal balloon; and
a flexible outer inflation shaft surrounding and coaxially aligned with the inner inflation shaft and having a second length shorter than the first length, a proximal end coupled to the proximal balloon inflation port, and a distal end coupled to the proximal balloon, the outer inflation shaft to carry a gas, liquid or solution that enters the proximal balloon inflation port and flows from the output port to the proximal balloon to inflate the proximal balloon.

2. The medical device of claim 1, wherein the distal balloon is positioned a third distance from the proximal balloon owing to the second length of the flexible outer inflation shaft being shorter than the first length of the flexible inner inflation shaft, wherein a segment of the flexible inner inflation shaft along a length of the third distance, or a portion thereof, allows for the flexible inner inflation shaft to bend or transition with respect to a position of the proximal balloon in a curved, U- or S-shaped configuration as the distal balloon, the flexible inner inflation shaft to which the distal balloon is coupled, and the proximal balloon traverse a vein or artery.

3. The medical device of claim 2, wherein the third distance is a fixed distance or a configurable distance.

4. The medical device of claim 3, wherein the segment comprises one or more markers affixed to the inner inflation shaft to indicate a location of the segment while inserted in a vein or artery of a body.

5. The medical device of claim 1, further comprising a coil coaxially aligned with and inside the flexible inner inflation shaft.

6. The medical device of claim 5, wherein the coil comprises a radioplaque material.

7. The medical device of claim 6, wherein both balloons can be independently inflated and deflated and either balloon can be interchangeably serves as occlusion balloon to block blood flow and to remove the implanted stents or other implanted material inside the arteries or vein the radioplaque material comprises platinum.

8. The medical device of claim 5, further comprising an innermost shaft coaxially aligned with and inside the inner inflation shaft, extending along the length of inner inflation shaft, wherein the innermost shaft houses the coil.

9. The medical device of claim 1, wherein the hub further comprises a guidewire port, through which a guidewire can be inserted to pass through the hub and output port and extend inside along the length of flexible inner inflation shaft.

10. The medical device of claim 1, further comprising one or more scoring wires affixed to an outer surface of one or both of the distal balloon and the proximal balloon so that when the gas, liquid or solution inflates one or both of the distal balloon and the proximal balloon, the one or more scoring wires engages and cuts through material in a vein or artery in which the distal balloon and proximal balloon is situated.

11. The medical device of claim 10, wherein the distal balloon is maintained in an inflated state to occlude blood flow while the scoring wires of the proximal balloon engage and cut through and loosen material adhering to the vein or artery to block the material from flowing past the distal balloon.

12. The medical device of claim 10, wherein the proximal balloon is maintained in an inflated state to occlude blood flow while the scoring wires of the distal balloon engage and cut through and loosen material adhering to the vein or artery to block the material from flowing past the proximal balloon.

13. The medical device of claim 10, wherein the scoring wire is selected from a group consisting of: an alloy of nickel titanium material, a metallic material, a composite material, and a polymer material.

14. The medical device of claim 1, further comprising one or more scoring wires affixed to an outer surface of a portion of the flexible inner inflation shaft between the distal balloon and the proximal balloon.

15. The medical device of claim 1, wherein the inner inflation shaft to carry the gas, liquid or solution that enters the distal balloon inflation port and flows from the output port to the distal balloon to inflate the distal balloon and the outer inflation shaft to carry the gas, liquid or solution, that enters the proximal balloon inflation port and flows from the output port to the proximal balloon to inflate the proximal balloon at concurrent, overlapping, or non-overlapping time periods.

16. The medical device of claim 1, wherein the guidewire input port functions as a dialysis output port, the hub further comprising a dialysis input port, and a return blood flow shaft coupled to the dialysis input port and affixed to an outer surface of the flexible outer inflation shaft, having a third length shorter than the second length and an return blood flow shaft output port via which blood flows in a vein.

17. The medical device of claim 1, wherein the distal balloon comprises a distal end and a proximal end that coincides with a respective pair of markers affixed to the inner inflation shaft to indicate a location of the distal balloon while inserted in a vein or artery of a body.

18. The medical device of claim 1, wherein the proximal balloon comprises a distal end and a proximal end that coincides with a respective pair of proximal balloon markers affixed to the outer inflation shaft to indication a location of the proximal balloon while inserted in a vein or artery of a body.

19. The medical device of claim 1, wherein the distal balloon and proximal balloon are inflated independent of each other.

20. The medical device of claim 1, wherein the distal balloon and the proximal balloon are inflated at one of simultaneous, concurrent, overlapping, and non-overlapping time periods.
